(19) United States
(12) Patent Application Publication
(10) Pub. No.: US 2022/0398711 A1
(43) Pub. Date: Dec. 15, 2022

(54) TRANSPARENCY DETECTION METHOD BASED ON MACHINE VISION

(71) Applicant: Zhejiang University, Hangzhou (CN)

(72) Inventors: Feng Lin, Hangzhou (CN); Qiannan Jin, Hangzhou (CN); Libo Gan, Hangzhou (CN)

(21) Appl. No.: 17/662,652

(22) Filed: May 9, 2022

Related U.S. Application Data
(63) Continuation of application No. PCT/CN2021/130867, filed on Nov. 16, 2021.

(30) Foreign Application Priority Data
May 26, 2021 (CN) 202110579783.3

(51) Int. Cl. G06T 7/00; G01N 33/18; G06T 7/11; G06T 7/70; G06V 10/82; G06T 7/60; G06V 20/40; G06T 7/194; G06T 7/136; G06V 10/762; H04N 5/232; G06V 10/764; G06T 3/40; G06V 10/774; G06T 3/60; G06V 30/146; G06T 7/13; G06V 30/19; G06V 30/14; G06T 7/90

(52) U.S. Cl. CPC G06T 7/0002; G01N 33/18; G06T 7/11; G06T 7/70; G06V 10/82; G06T 7/60; G06V 20/46; G06T 7/194; G06T 7/136; G06V 10/762; H04N 5/23245; G06V 10/764; G06T 3/40; G06V 10/774; G06T 3/60; G06V 30/1463; G06T 7/13; G06V 30/19173; G06V 30/1448; G06V 30/19107; G06V 20/41; G06T 7/90; G06T 2207/20084; G06T 2207/10016; G06T 2207/20021; G06T 2207/20081; G06T 2207/10024

(57) ABSTRACT

Disclosed is a transparency detecting method based on machine vision. The transparency detecting method includes 1) operating a Secchi disk to start the water transparency measurement, and turning on the camera for shooting; 2). determining a critical position of the Secchi disk; 3) identifying a water ruler and calculating a reading of the water ruler; 4) outputting and displaying the calculated reading.

### CROSS REFERENCE TO RELATED APPLICATIONS

This application is a continuation of PCT patent Application No. PCT/CN2021/130867, filed on 16 Nov. 2021, entitled “Transparency Detection Method Based on Machine Vision,” which claims foreign priority of Chinese Patent Application No. 202110579783.3, filed 26 May 2021 in the China National Intellectual Property Administration (CNIPA), the entire contents of which are hereby incorporated by reference in their entireties.

### FIELD OF THE DISCLOSURE

The disclosure relates to the field of computer vision, in particularly to a transparency detection method based on machine vision.

### BACKGROUND OF THE DISCLOSURE

Water transparency of bodies of water is a commonly used indicator to measure the water quality. A common method for measuring the transparency of water bodies is the employment of a Secchi disk. The employment of Secchi disk requires users to judge the ambiguity degree of the Secchi disk and read the depth of the water ruler. This is subject to the subjective factors of the observer, resulting in greater uncertainty in the measurement of transparency. In addition, external objective factors, such as the intensity of the light, the shake of the Secchi disk, etc., will also have an impact on the measurement of transparency. Such prior methods of transparency measurement require higher operator experience, and have greater uncertainty and instability. Now with the improvement of environmental and ecological requirements, water transparency has become a very important indicator. Accurate and real-time monitoring of changes in water transparency will help environmental protection departments make timely responses to urban river management. In the field of aquaculture, the level of water transparency will directly affect the production of aquaculture. Therefore, the accurate measurement of water body transparency is of great significance for promoting life and production.

There are a number of ways to measure transparency. For example, Chinese Patent Publication No. CN109859183A discloses a multi-element integrated water body intelligent identification method and an ecological station based on edge computing. In this method, the image of the target water area immersed in the Secchi disk is input into a preset SSD deep learning network, and based on the image recognition result of the Secchi disk output by the SSD deep learning network, the edge computing method is applied to obtain a current second water body transparence information in the target water area. However, this method needs to acquire at least one first water body transparency information and at least one first water level information of the target water area currently collected by the water body detection sensing component. The Chinese Patent Publication No. CN109784259A discloses an intelligent identification method of water body transparency based on image identification and a Secchi disk assembly. In the method, the image of the target water area immersed in the Secchi disk is input into a preset image recognition model, and the output of the image recognition model is used as the image recognition result of the Secchi disk. However, this method requires a plurality of disks including two-color working surfaces, and each of the disks is arranged around the cylinder in a spirally stepped arrangement. The Chinese Patent Publication No. CN110672563A discloses a method for image recognition and detection of transparency in smart water environment, which requires a special threaded rod with a Secchi disk to be vertically fixed at a water quality monitoring point. The above methods often need to obtain data in advance or require a specific device, the operation is relatively complicated, and the practicability is not strong.

### SUMMARY OF THE DISCLOSURE

With the tremendous development of computer vision technology, the use of computer vision technology to process image data can effectively avoid the influence of subjective and objective factors. Using computer vision technology to process image data has the advantages of high accuracy, wide application scenarios and strong generalization ability. This disclosure combines deep learning, image processing technology and Secchi disk measurement technology, and proposes an intelligent image processing and recognition technology based on machine vision for the measurement of water transparency.

One object of the present disclosure is to provide a transparency detection method based on machine vision. Number of the camera in the in the present disclosure may be one or two. When the water is clear, one camera may be used. When the transparency of the water quality is turbid, and one camera is not sufficient to track the movement of the Secchi disk and the water ruler at the same time, two cameras may be employed. One of the two cameras is used for taking pictures of the Secchi disk, and the other for the water ruler. The two cameras shoot at the same time, imitating the action of a person measuring the transparency of the water body with the Secchi disk (looking at the Secchi disk while looking at the water ruler), and obtain a clearer video of the Secchi disk and the water ruler. The captured video is transmitted to a computer for processing directly or through the cloud.

In order to achieve the above objects, the present disclosure includes two aspects. One of the two aspects is the judgment of the critical position of the Secchi disk, and the other is the identification of the water ruler. The schematic flowchart is shown in FIG. 1, and the specific steps include:

1) Operation of the Secchi disk to start the water transparency measurement, and turning on the camera for shooting;

2) Determination of a critical position of the Secchi disk;

3) Identification of the water ruler and calculation of the reading of the water ruler;

4) Output and display.

In step 2), the determination of the critical position of the Secchi disk: the critical position of the Secchi disk refers to a position when the Secchi disk is barely visible. It includes the steps of preliminary segmentation of the Secchi disk, fine segmentation of the Secchi disk, and determination of the critical position of the Secchi disk. More specifically:

2-1) preliminary segmentation of the Secchi disk: segmenting the white part of the Secchi disk from the video image; specifically includes steps: determining the Secchi disk size, positioning the Secchi disk in the image, and determining the threshold value.

2-1-1) Determining the Secchi disk size: in this disclosure, the Faster RCNN algorithm is used to identify the Secchi disk size:

It should be noted that the size here does not refer to the area of the Secchi disk, but an area of a rectangle. The four sides of the rectangle are fitted to enclose the Secchi disk. First, frame extraction is performed on the video containing the Secchi disk. In order to reduce the calculation amount of subsequent image processing, one image is captured every 3 frames in this disclosure. Then, the captured image is stored in a fixed folder. For the first image in the folder, using the Faster RCNN algorithm to identify the Secchi disk; the collected data is labeled with the image labeler function in Matlab2020b; the result of Faster RCNN identifying the Secchi disk size is shown in FIG. 2.

2-1-2) Positioning the Secchi disk in the image:

The position and size of the rectangular frame are obtained after recognizing the image of the Secchi disk in the initial position using the Faster RCNN. All images are divided into upper and lower parts with an upper edge of the rectangular frame being the dividing line. In this disclosure, starting from an upper left corner of the lower parts of the image, the previously determined rectangular frame is moved on the entire image with a certain step size; each time the rectangular frame is moved, the content in the rectangular frame is intercepted, and the average brightness in the frame is calculated. Because the white part on the Secchi disk has high brightness, the location of the sampling rectangular frame with a high average brightness value is the location of the Secchi disk.

When the image contains background objects whose brightness is higher than or similar to that of the Secchi disk, the background subtraction method based on the mean value is used in this disclosure: taking the images in the last few seconds of the video, there is no Secchi disk in the last few seconds of the video, so the last 10 frames of images of the video can be mean and taken as the background image; subtracting the background image from the images containing the Secchi disk, so the background part of the images becomes nearly 0 after subtraction; and then the location of the Secchi disk can be determined by the previous method in the subtracted image.

2-1-3) Determining the threshold

After positioning of the Secchi disk, extracting the Secchi disk from the original image, as shown in FIG. 3. Then, the segmented Secchi disk image is converted from RGB space to HSV space, and the luminance component is extracted to establish a luminance histogram, as shown in FIG. 4. The present disclosure uses the maximum inter-class variance method (Otsu's method) to determine the threshold, and the algorithm process is specified as follows:

Step one, recording the brightness value of the entire image as set C; the brightness values are divided into two categories, one is recorded as set C1, and the other is recorded as set C2, and C1∩C2=0, C1∪C2=C.

Step two, taking the brightness value k, and putting the brightness values in the range of [0, k−1] into the set C1, and put the remaining brightness values into the set C2. The mean value of the brightness values in the set C1 is recorded as m1, and the ratio of the element number in the set C1 to the element number in the set C is recorded as p1; the mean value of the brightness values in the set C2 is recorded as m2, and the ratio of element number in the set C2 to the element number of the set C is recorded as p2; the mean value of the brightness values in the set C is recorded as m; the calculation formula of the inter-class variance is recorded as: g=p1*(m1-m)∧2+p2*(m2-m)∧2.

Step three: the brightness value k is taken from 0 to 255 one by one; each time a value is taken, the corresponding maximum inter-class variance is calculated. The value k corresponding to the maximum inter-class variance is divided by 255 to obtain the final threshold.

After determining the threshold, keep all the brightness higher than the threshold, and remove the others, and then switch the image back to the RGB space to obtain the white part on the Secchi disk.

2-2) Fine segmentation of the Secchi disk: when the Secchi disk is almost invisible, the white part on the Secchi disk is segmented; in order to realize the fine segmentation of the Secchi disk, the present disclosure uses the following two steps:

2-2-1) Determining the segmentation threshold of the Secchi disk and segmenting it when the Secchi disk is almost invisible.

According to step 2-1), intercepting a Secchi disk video, using the method of step 2-1) to determine the threshold value of all the intercepted images, and creating a line graph of the threshold value of each image, as shown in FIG. 5.

Firstly, determine the position of the rectangular frame where the Secchi disk is located when the Secchi disk is very blurred. At the critical position, the Secchi disk is very blurred, and the Secchi disk will be invisible as long as the Secchi disk is descended a short distance. The diameter of a standard Secchi disk is 20 cm, and the short distance is much smaller than the diameter of the Secchi disk. Therefore, recording the height of the rectangular frame where the Secchi disk is located in the previous image of the transition point of the Secchi disk image as h, the width as w, the coordinates of the upper left corner vertex as [x, y], and using [x, y] as the starting point to create a rectangular area with a width of 1.6*w and a height of 1.6*h. This can ensure that the Secchi disk in the late images will appear in this rectangular area.

Then, use the threshold change trend to determine the threshold. Linear fitting is performed on the threshold data before the threshold transition point, and the fitted curve is used to predict the threshold after the transition point, and the predicted value is used as the segmentation threshold of the images after the transition point. For the determination of the transition point, the present disclosure adopts the K-means clustering analysis method. More specifically:

Step one, obtaining differences of the thresholds of adjacent points, and taking the absolute value of all differences;

Step two, using the K-means function that comes with MATLAB to classify these differences and divide them into two categories;

Step three, calculating the mean value of each category of differences, and take the category of difference with a greater mean value. The position where the category of difference first appears is the position of the transition point.

Finding the transition point position, and making the original threshold curve, threshold difference curve, and threshold fitting curve as shown in FIG. 6.

2-2-2) The critical threshold is determined by the background subtraction method based on the mean value; when the Secchi disk is completely invisible, the water is excluded from being segmented as Secchi disk.

In the present disclosure, the background subtraction method based on the mean value is used to obtain the background image. Each time the Secchi disk is segmented from the original image, the background image of the corresponding position is also segmented. Since the background images are all water, the background brightness values are in a normal distribution. Suggesting the mean value of the background brightness values is u, and the standard deviation is σ, and using u+2σ to replace the threshold value of the image to ensure that most of the parts will not be segmented when the Secchi disk is completely invisible in the image. This provides the basis for the determination of the critical position in step 2-3).

2-3) Determination of the critical position of the Secchi disk: using the classification network to determine the critical position of the Secchi disk; the specific steps are as follows:

2-3-1) Scaling up or down the segmentation results of all the white parts of the Secchi disk to a uniform size (e.g., 160*160*3). In the present disclosure, using the ResNet-18 network of MATLAB2020b to build a classification network to category the segmentation result of the Secchi disk. In this disclosure, the input of ResNet-18 is modified to a uniform size (e.g., 160*160*3 in this disclosure), and the parameters of each layer of the network are shown in Table 1.

2-3-2) Collecting more Secchi disk videos and create a dataset for classification network training. The data in this disclosure is divided into two categories, one for having Secchi disk, and the other without Secchi disk. Some of the dataset image is shown in FIG. 7.

2-3-3) Using the trained classification network to determine the critical position of the Secchi disk.

2-3-4) After obtaining the critical position of the Secchi disk, performing correction on the critical position. Due to the perspective distance difference between human eyes and the camera, and the accuracy of the current cameras are getting higher and higher, there is an error between the eyes and the camera. In fact, the judgment distance between people, the judgment distance of each person in different states is also different. Therefore, a standard is needed, and the camera as a standard is undoubtedly relatively more objective. However, the error is relatively fixed. Providing the error is ΔD, then the actual critical position of the Secchi disk is the critical position of the Secchi disk+ΔD.

3) Recognition of the water ruler and calculation of the water ruler reading. In this disclosure, the Deeplabv3+ algorithm is used to identify and segment the water ruler at the position, then extracting the characters on the water ruler, classifying the characters, and calculating the water ruler reading to obtain the transparency value. More specifically:

3-1) Using the Deeplabv3+ algorithm to identify and segment the water ruler at the position; comprising the following steps:

3-1-1) Creating a data set; in this example; more than 120 water ruler images are collected as a training data set, and the Image Labeler function that comes with MATLAB2020b is used for data labeling. Some of the water ruler images and the labeling results are shown in FIG. 8.

3-1-2) Using Deeplabv3+ for network training;

3-1-3) Using the trained Deeplabv3+ for water ruler segmentation.

3-2) Extracting the characters on the water ruler; including the following steps:

3-2-1) Tilt correction of water ruler.

The result of Deeplabv3+ segmentation is tilted by least squares estimation, and the calculation formula is shown in formula (1):

$$a = \frac{n \sum {x}_{i} {y}_{i} - \sum {x}_{i} \sum {y}_{i}}{n \sum {x}_{i}^{2} - \sum {x}_{i} \sum {x}_{i}} \qquad ( 1 )$$

Let the coordinates of the point on the left or right edge of the water ruler be (xi, yi), where xi represents the abscissa, yi represents the ordinate, i ∈ [1, n], and calculate the slope of the water ruler.

3-2-2) Determine the edge position of the corrected water ruler, and then segment the water ruler from the original image.

3-2-3) Transpose and mirror the segmented water ruler to make the segmented water ruler rotate 90 degrees to the right to become a horizontal water ruler.

3-2-4) Use the im2bw function of MATLAB to binarize the segmented water ruler, invert the binarized image, and use the corrosion algorithm to disconnect the characters.

3-2-5) Use the regionprops( )function in MATLAB to mark all characters with a rectangular box, as shown in FIG. 9.

3-2-6) Use K-means clustering analysis to separate large characters from small characters.

After the above steps, the valid characters (larger characters) to be obtained in this disclosure are still retained in the image, while the other characters (small characters) are either eroded or retained. In this disclosure, the K-means clustering algorithm is used to separate large characters from small characters. More specifically as follows:

Step one, calculating the area of the rectangular frame surrounding the character.

Step two, using the kmeans( )function in MATLAB to perform clustering analysis on the area of said rectangular frame, and divide the areas of rectangular frames into two categories.

Step three, calculating the mean value of each category. The category with the greater mean value is the large characters, and the large category having the large character is segmented. The large characters obtained by clustering are shown in FIG. 10.

3-3) Classify the characters of the water ruler; in this disclosure, a CNN classification network is constructed to classify the characters of the water ruler, and the ResNet-18 network of MATLAB2020b is used as the classifier. A total of 10 classes are set, and each number from 0 to 9 belongs to one class. This disclosure uses the digital character data set in MATLAB, binarizes all the images in the data set, and scales them to a size of 64*64*1.

3-4) Calculate the reading of the water ruler to get the transparency value; more specifically as follows:

For a horizontal water ruler, the position of each full ten scale is located between each non-zero number and the number 0 immediately to the right of non-zero number. For example, the position of the mark 70 is between the character 7 and the character 0 to the right of the character 7. Let the non-zero number be k, the position of the right edge of the number k is recorded as x_right(k); the position of the left edge of the number 0 immediately to the right of the number k is recorded as x_left(k); then the position of each full ten scale is x(k) is calculated as shown in (2):

$$x ( k ) = \frac{x\_left ( k ) + x\_right ( k )}{2} \qquad ( 2 )$$

Marking the minimum non-zero number on the water ruler as i, then the calculation formula of the reading of the water ruler can be obtained according to the ratio relationship as shown in (3):

$$value = 10 * ( i - \frac{x ( i )}{x ( i + 1 ) - x ( i )} ) \qquad ( 3 )$$

4) Output and display the reading of the water ruler.

Compared with the prior art, the advantages of the present disclosure are as follows.

The above technical solution proposes an image processing-based intelligent Secchi disk and water ruler identification technology, which combines traditional Secchi disk with image processing, deep learning and other technologies to accurately measure the transparency of water bodies and overcome the need for manual measurement. Due to subjective and objective factors in the process, there are errors in readings and inaccurate disc position judgment. The method in the present disclosure has high accuracy, stable and objective numerical values, and is not affected by individual subjective factors, and has high application value.

### BRIEF DESCRIPTION OF DRAWINGS

FIG. 1 is a schematic flowchart of a method for detecting transparency based on machine vision in one embodiment of the present disclosure.

FIG. 2 is the result diagram of adopting faster R-CNN to identify the size of Secchi disk in the embodiment of the present disclosure.

FIG. 3 is the Secchi disk that is segmented in the embodiment of the present disclosure.

FIG. 4 is the brightness histogram of the Secchi disk that is segmented in the embodiment of the present disclosure;

FIG. 5 is a threshold line graph according to one embodiment of the present disclosure.

FIG. 6 is an original threshold curve, a threshold difference curve, and a threshold fitting curve diagram according to one embodiment of the present disclosure.

FIG. 7 is a schematic diagram of a partial data set of ResNet-18 in one embodiment of the present disclosure.

FIG. 8 is some water rulers and their labeling result images in the embodiment of the present disclosure.

FIG. 9A and FIG. 9B shows the result of the water ruler image processing in the embodiment of the present disclosure, wherein FIG. 9A is the horizontal water ruler image, and FIG. 9B is the water ruler image after binarization, inversion and corrosion.

FIG. 10 shows large characters obtained by clustering in an embodiment of the present disclosure.

### DETAILED DESCRIPTION OF THE DISCLOSURE

In order to make the objectives, technical solutions and advantages of the present disclosure clearer, the present disclosure will be further described below with reference to the embodiments and the accompanying drawings. Obviously, the described embodiments are some, but not all, embodiments of the present disclosure. Based on the described embodiments, all other embodiments obtained by those of ordinary skill in the art without creative efforts fall within the protection scope of the present disclosure.

Unless otherwise defined, technical or scientific terms used in the present disclosure should have the ordinary meaning as understood by one of ordinary skill in the art to which the present disclosure belongs. When used herein, “comprising” or “including” and similar words mean that the elements or things appearing before the word encompass the elements or things listed after the word and their equivalents, but do not exclude other elements or things. Words like “connected” or “connected” are not limited to physical or mechanical connections, but may include electrical connections, whether direct or indirect. Terms “up”, “down”, “left”, “right”, etc. are only used to represent the relative positional relationship, and when the absolute position of the described object changes, the relative positional relationship may also change accordingly.

### Embodiment

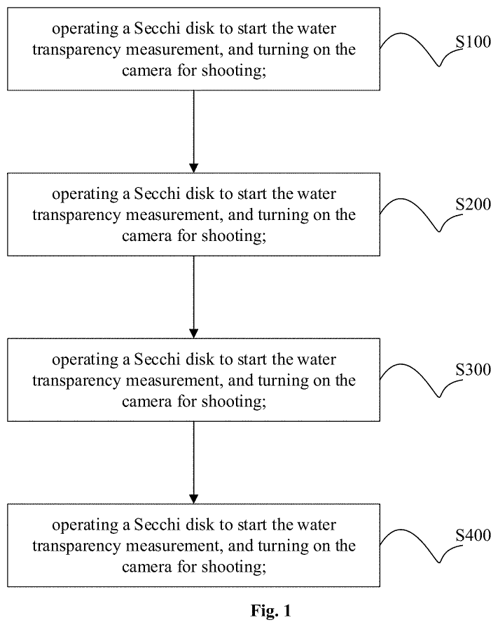

Referring to FIG. 1 to FIG. 10.

One object of the present disclosure is to provide a transparency detection method based on machine vision. Number of the camera in the in the present disclosure may be one or two. When the water is clear, one camera may be used. When the transparency of the water quality is turbid, and one camera is not sufficient to track the movement of the Secchi disk and the water ruler at the same time, two cameras may be employed. One of the two cameras is used for taking pictures of the Secchi disk, and the other for the water ruler. The two cameras shoot at the same time, imitating the action of a person measuring the transparency of the water body with the Secchi disk (looking at the Secchi disk while looking at the water ruler), and obtain a clearer video of the Secchi disk and the water ruler. The captured video is transmitted to a computer for processing directly or through the cloud. In order to achieve the above objects, the present disclosure includes two aspects. One of the two aspects is the judgment of the critical position of the Secchi disk, and the other is the identification of the water ruler. The schematic flowchart is shown in FIG. 1, and the steps is specified as follows.

S100, operating the Secchi disk to start the water transparency measurement, and turning on the camera for shooting.

S200, determining a critical position of the Secchi disk; the critical position of the Secchi disk refers to a position when the Secchi disk is barely visible. The step of determining the critical position of the Secchi disk includes the steps of preliminary segmentation of the Secchi disk, fine segmentation of the Secchi disk, and determination of the critical position of the Secchi disk. More specifically as follows.

S210, preliminary segmentation of the Secchi disk: segmenting the white part of the Secchi disk from the video image; specifically includes steps: determining the Secchi disk size, positioning the Secchi disk in the image, and determining the threshold value.

S211, determining the Secchi disk size: in this disclosure, the Faster RCNN algorithm is used to identify the Secchi disk size. First, frame extraction is performed on the video containing the Secchi disk. In order to reduce the calculation amount of subsequent image processing, one image is captured every 3 frames in this disclosure. Then, the captured image is stored in a fixed folder. For the first image in the folder, using the Faster RCNN algorithm to identify the Secchi disk; the collected data is labeled with the image labeler function in Matlab2020b; the result of Faster RCNN identifying the Secchi disk size is shown in FIG. 2.

S212, Positioning the Secchi disk in the image.

The position and size of the rectangular frame are obtained after recognizing the image of the Secchi disk in the initial position using the Faster RCNN. All images are divided into upper and lower parts with an upper edge of the rectangular frame being the dividing line. In this disclosure, starting from an upper left corner of the lower parts of the image, the previously determined rectangular frame is moved on the entire image with a certain step size; each time the rectangular frame is moved, the content in the rectangular frame is intercepted, and the average brightness in the frame is calculated. Because the white part on the Secchi disk has high brightness, the location of the sampling rectangular frame with a high average brightness value is the location of the Secchi disk.

When the image contains background objects whose brightness is higher than or similar to that of the Secchi disk, the background subtraction method based on the mean value is used in this disclosure: taking the images in the last few seconds of the video, there is no Secchi disk in the last few seconds of the video, so the last 10 frames of images of the video can be mean and taken as the background image; subtracting the background image from the images containing the Secchi disk, so the background part of the images becomes nearly 0 after subtraction; and then the location of the Secchi disk can be determined by the previous method in the subtracted image.

S213, Determining the threshold.

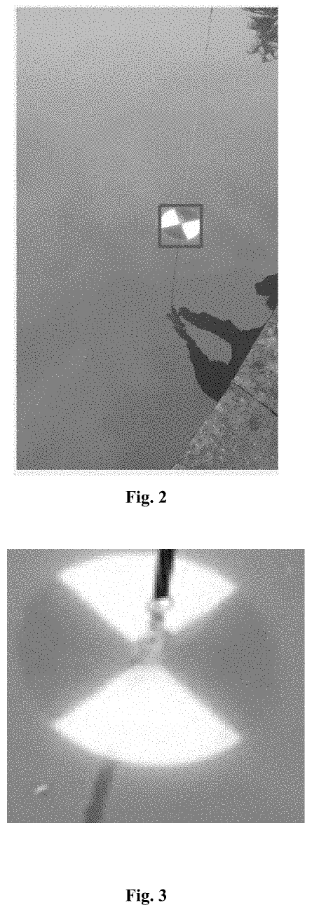

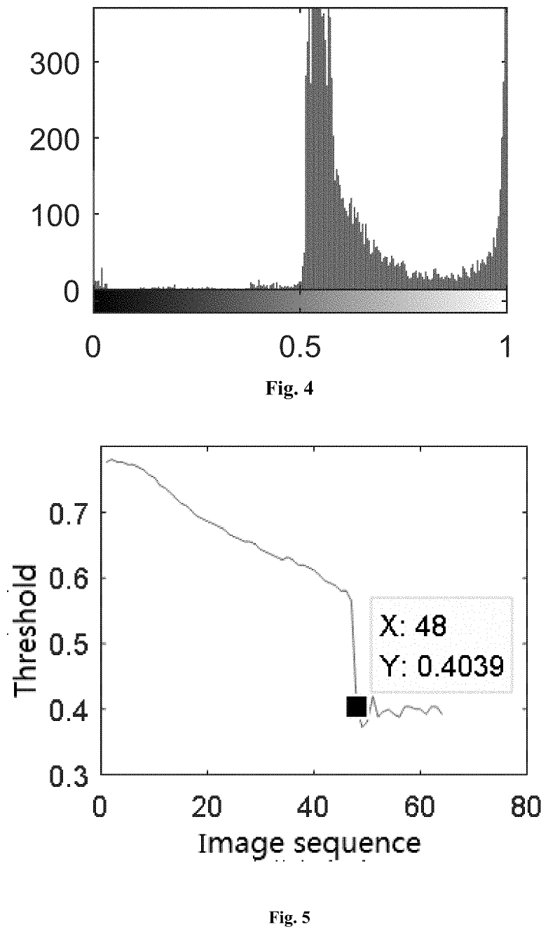

After positioning of the Secchi disk, extracting the Secchi disk from the original image, as shown in FIG. 3. Then, the segmented Secchi disk image is converted from RGB space to HSV space, and the luminance component is extracted to establish a luminance histogram, as shown in FIG. 4. The present disclosure uses the maximum inter-class variance method (Otsu's method) to determine the threshold, and the algorithm process is specified as follows.

Step one, recording the brightness value of the entire image as set C; the brightness values are divided into two categories, one is recorded as set C1, and the other is recorded as set C2, and C1∩C2=0, C1∪C2=C.

Step two, taking the brightness value k, and putting the brightness values in the range of [0, k−1] into the set C1, and put the remaining brightness values into the set C2. The mean value of the brightness values in the set C1 is recorded as m1, and the ratio of the element number in the set C1 to the element number in the set C is recorded as p1; the mean value of the brightness values in the set C2 is recorded as m2, and the ratio of element number in the set C2 to the element number of the set C is recorded as p2; the mean value of the brightness values in the set C is recorded as m; the calculation formula of the inter-class variance is recorded as: g=p1*(m1−m)∧2+p2*(m2−m)∧2.

Step three: the brightness value k is taken from 0 to 255 one by one; each time a value is taken, the corresponding maximum inter-class variance is calculated. The value k corresponding to the maximum inter-class variance is divided by 255 to obtain the final threshold.

After determining the threshold, keep all the brightness higher than the threshold, and remove the others, and then switch the image back to the RGB space to obtain the white part on the Secchi disk.

S220, Fine segmentation of the Secchi disk. When the Secchi disk is almost invisible, the white part on the Secchi disk is segmented. In order to realize the fine segmentation of the Secchi disk, the present disclosure uses the following two steps:

S221, Determining the segmentation threshold of the Secchi disk and segmenting The Secchi disk when the Secchi disk is almost invisible.

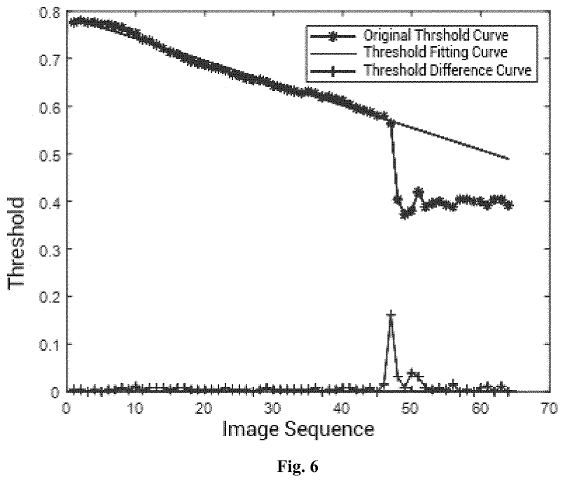

According to step S210, intercepting a Secchi disk video, using the method of step 2-1) to determine the threshold value of all the intercepted images, and creating a line graph of the threshold value of each image, as shown in FIG. 5.

Firstly, determine the position of the rectangular frame where the Secchi disk is located when the Secchi disk is very blurred. At the critical position, the Secchi disk is very blurred, and the Secchi disk will be invisible as long as the Secchi disk is descended a short distance. The diameter of a standard Secchi disk is 20 cm, and the short distance is much smaller than the diameter of the Secchi disk. Therefore, recording the height of the rectangular frame where the Secchi disk is located in the previous image of the transition point of the Secchi disk image as h, the width as w, the coordinates of the upper left corner vertex as [x, y], and using [x, y] as the starting point to create a rectangular area with a width of 1.6*w and a height of 1.6*h. This can ensure that the Secchi disk in the late images will appear in this rectangular area.

Then, use the threshold change trend to determine the threshold. Linear fitting is performed on the threshold data before the threshold transition point, and the fitted curve is used to predict the threshold after the transition point, and the predicted value is used as the segmentation threshold of the images after the transition point. For the determination of the transition point, the present disclosure adopts the K-means clustering analysis method. The K-means clustering analysis method is specified as follows.

Step one, obtaining differences of the thresholds of adjacent points, and taking the absolute value of all differences.

Step two, using the K-means function that comes with MATLAB to classify these differences and divide them into two categories.

Step three, calculating the mean value of each category of differences, and take the category of difference with a greater mean value. The position where the category of difference first appears is the position of the transition point.

Finding the transition point position, and making the original threshold curve, threshold difference curve, and threshold fitting curve as shown in FIG. 6.

S222, determining the critical threshold by the background subtraction method based on the mean value; when the Secchi disk is completely invisible, the water is excluded from being segmented as Secchi disk.

In the present disclosure, the background subtraction method based on the mean value is used to obtain the background image. Each time the Secchi disk is segmented from the original image, the background image of the corresponding position is also segmented. Since the background images are all water, the background brightness values are in a normal distribution. Suggesting the mean value of the background brightness values is u, and the standard deviation is σ, and using u+2σ to replace the threshold value of the image to ensure that most of the parts will not be segmented when the Secchi disk is completely invisible in the image. This provides the basis for the determination of the critical position in S230.

S230, determining the critical position of the Secchi disk: using the classification network to determine the critical position of the Secchi disk. The specific steps for S230 are as follows.

S231, scaling up or down the segmentation results of all the white parts of the Secchi disk to a uniform size (e.g., 160*160*3). In the present disclosure, using the ResNet-18 network of MATLAB2020b to build a classification network to category the segmentation result of the Secchi disk. In this disclosure, the input of ResNet-18 is modified to a uniform size (e.g., 160*160*3 in this disclosure), and the parameters of each layer of the network are shown in Table 1.

TABLE 1
Parameters in each ResNet-18 layer

| Layer Name | Output Size of Layer | Parameter |
|---|---|---|
| Conv1 | 80*80 | 7*7, 64, stride 2 |
| Conv2_x | 40*40 | 3*3, max pool, stride 2 |
| | | $[ \begin{matrix}3 * 3 , & 64 \\ 3 * 3 , & 64\end{matrix} ] * 2$ |
| Conv3_x | 20*20 | $[ \begin{matrix}3 * 3 , & 128 \\ 3 * 3 , & 128\end{matrix} ] * 2$ |
| Conv4_x | 10*10 | $[ \begin{matrix}3 * 3 , & 256 \\ 3 * 3 , & 256\end{matrix} ] * 2$ |
| Conv5_x | 5*5 | $[ \begin{matrix}3 * 3 , & 512 \\ 3 * 3 , & 512\end{matrix} ] * 2$ |
| | 1*1 | average pool, fc, softmax |

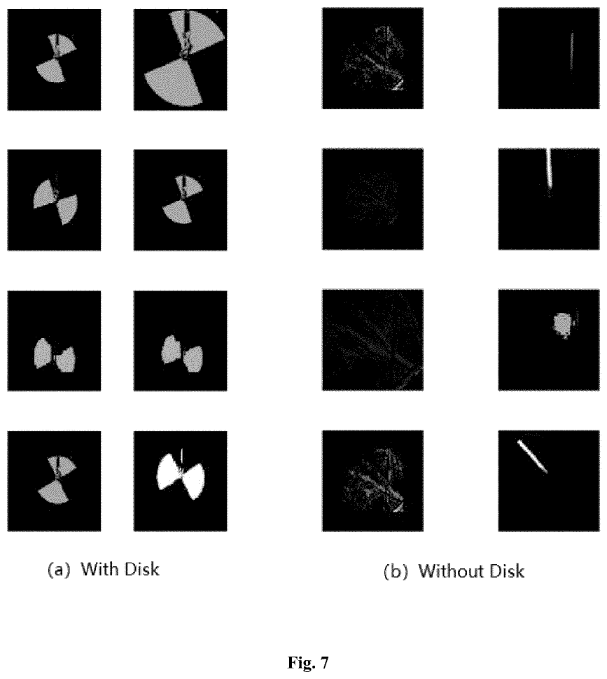

S232, collecting more Secchi disk videos and create a dataset for classification network training. The data in this disclosure is divided into two categories, one for having Secchi disk, and the other without Secchi disk. Some dataset image is shown in FIG. 7.

S233, using the trained classification network to determine the critical position of the Secchi disk.

S234, after obtaining the critical position of the Secchi disk, performing correction on the critical position. Due to the perspective distance difference between human eyes and the camera, and the accuracy of the current cameras are getting higher and higher, there is an error between the eyes and the camera. In fact, the judgment distance between people, the judgment distance of each person in different states is also different. Therefore, a standard is needed, and the camera as a standard is undoubtedly relatively more objective. However, the error is relatively fixed. Providing the error is ΔD, then the actual critical position of the Secchi disk is +ΔD. In the present disclosure, ΔD=−5 cm.

S300, identifying the water ruler and calculating the water ruler reading. In this disclosure, the Deeplabv3+ algorithm is used to identify and segment the water ruler at the position, then extracting the characters on the water ruler, classifying the characters, and calculating the water ruler reading to obtain the transparency value. More specifically as follows.

S310, using the Deeplabv3+ algorithm to identify and segment the water ruler at the position; comprising the following steps:

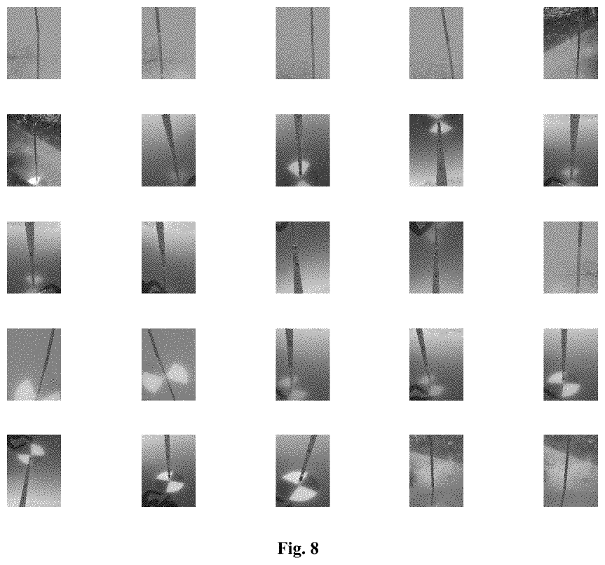

S311, creating a data set; in this example; more than 120 water ruler images are collected as a training data set, and the Image Labeler function that comes with MATLAB2020b is used for data labeling. Some of the water ruler images and the labeling results are shown in FIG. 8.

S312, using Deeplabv3+ for network training;

S313, using the trained Deeplabv3+ for water ruler segmentation.

S320, extracting the characters on the water ruler. The step of S320 includes the following steps.

S321, Tilt correction of water ruler. The result of Deeplabv3+ segmentation is tilted by least squares estimation, and the calculation formula is shown in formula (1):

$$a = \frac{n \sum {x}_{i} {y}_{i} - \sum {x}_{i} \sum {y}_{i}}{n \sum {x}_{i}^{2} - \sum {x}_{i} \sum {x}_{i}} \qquad ( 1 )$$

Let the coordinates of the point on the left or right edge of the water ruler be (xi, yi), where xi represents the abscissa, yi represents the ordinate, i ∈ [1, n], and calculate the slope of the water ruler.

S322, determining the edge position of the corrected water ruler, and then segmenting the water ruler from the original image.

S323, transposing and mirroring the segmented water ruler to make the segmented water ruler rotate 90 degrees to the right to become a horizontal water ruler.

S324, using the im2bw function of MATLAB to binarize the segmented water ruler, inverting the binarized image, and using the corrosion algorithm to disconnect the characters.

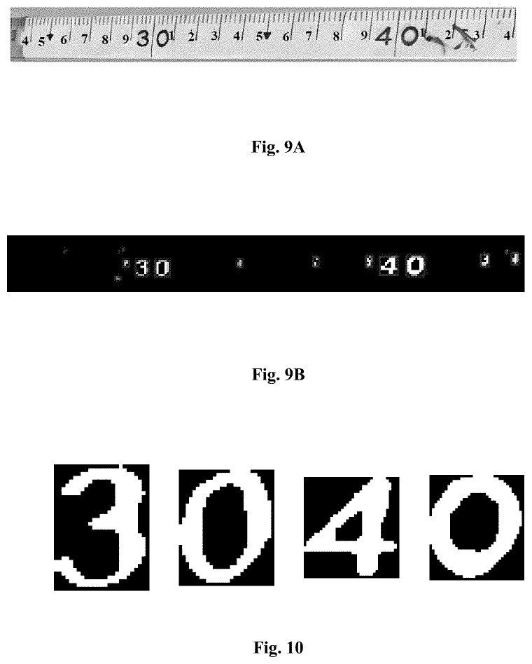

S325, using the regionprops( ) function in MATLAB to mark all characters with a rectangular box, as shown in FIG. 9.

S326, using K-means clustering analysis to separate large characters from small characters.

After the above steps, the valid characters (larger characters) to be obtained in this disclosure are still retained in the image, while the other characters (small characters) are either eroded or retained. In this disclosure, the K-means clustering algorithm is used to separate large characters from small characters. More specifically as follows:

Step one, calculating the area of the rectangular frame surrounding the character.

Step two, using the kmeans( ) function in MATLAB to perform clustering analysis on the area of said rectangular frame, and divide the areas of rectangular frames into two categories.

Step three, calculating the mean value of each category. The category with the greater mean value is the large characters, and the large category having the large character is segmented. The large characters obtained by clustering are shown in FIG. 10.

S330, Classifying the characters of the water ruler. In this disclosure, a CNN classification network is constructed to classify the characters of the water ruler, and the ResNet-18 network of MATLAB2020b is used as the classifier. A total of 10 classes are set, and each number from 0 to 9 belongs to one class. This disclosure uses the digital character data set in MATLAB, binarizes all the images in the data set, and scales them to a size of 64*64*1.

S340, calculating the reading of the water ruler to get the transparency value. more specifically as follows.

For a horizontal water ruler, the position of each full ten scale is located between each non-zero number and the number 0 immediately to the right of non-zero number. For example, the position of the mark 70 is between the character 7 and the character 0 to the right of the character 7. Let the non-zero number be k, the position of the right edge of the number k is recorded as x_right(k); the position of the left edge of the number 0 immediately to the right of the number k is recorded as x_left(k); then the position of each full ten scale is x(k) is calculated as shown in (2):

$$x ( k ) = \frac{x\_left ( k ) + x\_right ( k )}{2} \qquad ( 2 )$$

Marking the minimum non-zero number on the water ruler as i, then the calculation formula of the reading of the water ruler can be obtained according to the ratio relationship as shown in (3):

$$value = 10 * ( i - \frac{x ( i )}{x ( i + 1 ) - x ( i )} ) \qquad ( 3 )$$

S400, Output and display the reading of the water ruler.

What is claimed is:

1. A transparency detecting method based on machine vision, comprising:
1) operating a Secchi disk to start the water transparency measurement, and turning on the camera for shooting;
2) determining a critical position of the Secchi disk;
3) identifying a water ruler and calculating a reading of the water ruler;
4) outputting and displaying the calculated reading;
wherein the step 2) determining the critical position of the Secchi disk comprises: a preliminary segmentation of the Secchi disk, a fine segmentation of the Secchi disk, and a determination of the critical position of the Secchi disk;
2-1) preliminary segmentation of the Secchi disk: segmenting white parts of the Secchi disk from a video image; the step of segmenting the white parts of the Secchi disk from the video image comprises: determining a Secchi disk size, positioning the Secchi disk in the image, and determining a threshold value;
2-1-1) determining the Secchi disk size, wherein a Faster RCNN algorithm is used to identify the Secchi disk size; the Secchi disk size refers to an area of a rectangle, wherein four sides of the rectangle are fitted to enclose the Secchi disk; firstly, performing frame extraction on a video containing the Secchi disk; one image is captured every 3 frames; the captured image is stored in a fixed folder; for the first image in the folder, using the Faster RCNN algorithm to identify the Secchi disk; collected data of the identified Secchi disk is labeled with Image Labeler function in MATLAB2020b;
2-1-2) positioning the Secchi disk in the image; wherein the position and size of a rectangular frame are obtained after recognizing the image of the Secchi disk in the initial position using the Faster RCNN; the image is divided into an upper part and a lower part using an upper edge of the rectangular frame being the dividing line; starting from an upper left corner of the lower part of the image, the preset rectangular frame is moved on the image with a certain step size; each time the rectangular frame is moved, content in the rectangular frame is intercepted, and a brightness mean in the rectangular frame is calculated; location of the rectangular frame with a high average brightness value is the location of the Secchi disk;
when the image contains background objects whose brightness is higher than or similar to that of the Secchi disk, averaging last 10 frames of images of the video and taking the averaged image as a background image; subtracting the background image from the images containing the Secchi disk, so the background part of the images becomes nearly 0 after the subtraction; and then the location of the Secchi disk is determined by performing the step 2-1-2) on the substrate image;
2-1-3) determining the threshold; after positioning of the Secchi disk, extracting the Secchi disk from a original image; then, converting the segmented Secchi disk image from RGB space to HSV space, and extracting a luminance component from the converted image to establish a luminance histogram; using a maximum inter-class variance method (Otsu's method) to determine the threshold; the step of using a maximum inter-class variance method (Otsu's method) to determine the threshold comprises the following steps:
step one, recording brightness value of the entire image as set C; the brightness values are divided into two categories, one is recorded as set C1, and the other is recorded as set C2, and C2, and C1∩C2=0, C1∪C2=C;
step two, taking a brightness value k, and putting the brightness values in the range of [0, k−1] into the set C1, and putting the remaining brightness values into the set C2; a mean value of the brightness values in the set C1 is recorded as m1, and a ratio of element number in the set C1 to element number in the set C is recorded as p1; a mean value of the brightness values in the set C2 is recorded as m2, and a ratio of element number in the set C2 to element number of the set C is recorded as p2; the mean value of the brightness values in the set C is recorded as m; the calculation formula of the inter-class variance is recorded as: g=p1*(m1−m)∧2+p2*(m2−m)∧2;
step three, taking the brightness value k from 0 to 255 one by one; each time a value is taken, the corresponding maximum inter-class variance is calculated; the brightness value k corresponding to the maximum inter-class variance is divided by 255 to obtain a final threshold.
after determining the threshold, keeping all the brightness higher than the threshold, and removing those lower or equal to the threshold; and then switching the image back to the RGB space to obtain the white parts on the Secchi disk;
2-2) fine segmentation of the Secchi disk: when the Secchi disk is almost invisible, segmenting the white parts on the Secchi disk; the step of fine segmentation of the Secchi disk comprising the following steps:
2-2-1) determining the segmentation threshold of the Secchi disk and segmenting Secchi disk when the Secchi disk is approaching the critical position;
according to step 2-1), intercepting a Secchi disk video, using the method of step 2-1) to determine the threshold values of all the intercepted images, and creating a line graph of the threshold values of each image;
firstly, determining a position of the rectangular frame where the Secchi disk is located when the Secchi disk is approaching the critical position; providing a height of the rectangular frame where the Secchi disk is located in a previous image of a transition point of the Secchi disk image is h, a width w, a coordinate of the upper left corner vertex [x, y]; and using [x, y] as a starting point to create a rectangular area with a width of 1.6*w and a height of 1.6*h, ensuring the Secchi disk in the late images to appear in the rectangular area;
then, using a threshold change trend to determine the threshold; performing linear fitting on a threshold data before the threshold transition point, and using a fitted curve to predict the threshold after the transition point, the predicted value being a segmentation threshold of the images after the transition point; the transition point is determined by a K-means clustering analysis method, comprising the following steps:
step one, obtaining differences of the thresholds of adjacent points, and taking a absolute value of the differences;
step two, using a K-means function in MATLAB to classify the differences and divide the classified differences into two categories comprising a first category having differences of threshold transition positions, and a second category having differences of non-transition positions;
step three, calculating a mean value of the first category and the second category, and taking either of the first category or the second category having a greater mean value for calculation; a position where the first or second category having the greater mean value first appears is the position of the transition point;
finding the transition point position, and making a original threshold curve, a threshold difference curve, and a threshold fitting curve;
2-2-2) determining the critical threshold by the background subtraction method based on the mean value; when the Secchi disk is invisible, the water is excluded from being segmented as Secchi disk; the background subtraction method based on the mean value is used to obtain the background image; each time the Secchi disk is segmented from the original image, the background image of the corresponding position is also segmented; since the background images are all water, background brightness values are in a normal distribution; providing the mean value of the background brightness values is u, and the standard deviation is σ, and the threshold value of the background image is u+2 σ;
2-3) determination of the critical position of the Secchi disk: using a classification network to determine the critical position of the Secchi disk; the step of using the classification network to determine the critical position of the Secchi disk comprising steps as follows:
2-3-1) scaling up or down segmentation results of all the white parts of the Secchi disk to a uniform size (e.g., 160*160*3); wherein ResNet-18 network of MATLAB2020b is used to build a classification network to category the segmentation results of the Secchi disk;
2-3-2) collecting more Secchi disk videos and creating a dataset for classification network training; the collected data is divided into two categories including a Secchi disk data and a non-Secchi disk data;
2-3-3) using the trained classification network to determine the critical position of the Secchi disk;
2-3-4) after obtaining the critical position of the Secchi disk, performing correction on the critical position; an actual critical position of the Secchi disk is: the critical position of the Secchi disk+ΔD, wherein ΔD is a difference caused by eyes and cameras.

2. The method of claim 1, wherein the step 3) identifying the water ruler and calculating the water ruler reading comprising: using a Deeplabv3+ algorithm identify and segment the water ruler at the actual critical position, then extracting characters on the water ruler, classifying the characters, and calculating the water ruler reading to obtain the transparency value; wherein:
3-1) using the Deeplabv3+ algorithm to identify and segmenting the water ruler at the actual critical position; comprising the following steps:
3-1-1) creating a data set; using an Image Labeler function in MATLAB2020b for data labeling;
3-1-2) using Deeplabv3+ for network training;
3-1-3) using the trained Deeplabv3+ for water ruler segmentation;
3-2) extracting the characters on the water ruler; the step of extracting the characters on the water ruler includes the following steps:
3-2-1) performing tilt correction for the water ruler;
using least squares estimation to perform tilt correction for result of Deeplabv3+ segmentation, and the calculation formula is shown in formula (1):
$$a = \frac{n \sum {x}_{i} {y}_{i} - \sum {x}_{i} \sum {y}_{i}}{n \sum {x}_{i}^{2} - \sum {x}_{i} \sum {x}_{i}} \qquad ( 1 )$$
providing coordinates of point on the left or right edge of the water ruler be (xi, yi), where xi represents the abscissa, yi represents the ordinate, i ∈ [1, n], and calculating a slope of the water ruler;
3-2-2) determining edge position of the corrected water ruler, and then segmenting the water ruler from the original image;
3-2-3) transposing and mirroring the segmented water ruler to make the segmented water ruler rotate 90 degrees to the right to become a horizontal water ruler;
3-2-4) using an im2bw function in MATLAB to binarize the segmented water ruler, inverting the binarized image, and using the corrosion algorithm to disconnect the characters;
3-2-5) using the regionprops( ) function in MATLAB to mark all characters with a rectangular box;
3-2-6) using K-means clustering analysis to separate large characters from small characters;
the step using K-mean clustering analysis comprises the following steps:
step one, calculating an area of the rectangular box surrounding the character. step two, using the kmeans( ) function in MATLAB to perform clustering analysis on the area of said rectangular box, and divide the areas of the rectangular boxes into two categories;
step three, calculating a mean value of the two categories; the category with the greater mean value is defined as a large character category, and the large character category having the large character is segmented;
3-3) classifying the characters of the water ruler; wherein a CNN classification network is constructed to classify the characters of the water ruler, and a ResNet-18 network in MATLAB2020b is used as the classifier; a total of 10 classes are set, and each number from 0 to 9 belongs to one class; digital character data set in MATLAB is used, and the data set in all images is binarized and scaled to a uniform size;
3-4) calculating the reading of the water ruler to obtain the transparency value;
for a horizontal water ruler, the position of each full ten scale is located between each non-zero number and the number 0 immediately to the right of non-zero number; providing the non-zero number be k, the position of the right edge of the number k is recorded as x_right(k); the position of the left edge of the number 0 immediately to the right of the number k is recorded as x_left(k); then the position of each full ten scale is x(k) is calculated as shown in (2):
$$x ( k ) = \frac{x\_left ( k ) + x\_right ( k )}{2} ; \qquad ( 2 )$$
marking the minimum non-zero number on the water ruler as i, then the calculation formula of the reading of the water ruler can be obtained according to the ratio relationship as shown in (3):
$$value = 10 * ( i - \frac{x ( i )}{x ( i + 1 ) - x ( i )} ) . \qquad ( 3 )$$
